(19) United States
(12) Patent Application Publication
(10) Pub. No.: US 2022/0096169 A1
(43) Pub. Date: Mar. 31, 2022

(54) TRACKING OF INSTRUMENT MOTIONS USING AN INERTIAL MEASUREMENT SYSTEM

(71) Applicant: CARNEGIE MELLON UNIVERSITY, Pittsburgh, PA (US)

(72) Inventors: Ernest Kabuye, Pittsburgh, PA (US); Philip LeDuc, Wexford, PA (US); Jonathan Cagan, Pittsburgh, PA (US); Carmel Majidi, Pittsburgh, PA (US)

(21) Appl. No.: 17/486,419

(22) Filed: Sep. 27, 2021

Related U.S. Application Data
(60) Provisional application No. 63/084,952, filed on Sep. 29, 2020.

(51) Int. Cl. A61B 34/20; A61B 34/10; A61B 34/30; A61B 17/3211

(52) U.S. Cl. CPC A61B 34/20; A61B 34/10; A61B 34/30; A61B 17/3211; A61B 2017/00526; A61B 2562/166; A61B 2562/12; A61B 2562/028; A61B 2034/2074; A61B 2562/0219

(57) ABSTRACT

Disclosed herein is system, including a hand-held tool, for example, a surgical scalpel, integrated with a 9 degree-of-freedom inertial measurement unit and a method for tracking the location of the hand-held instrument during manual or robotically-assisted procedures. The system and method has application in the surgical field, wherein instrumented surgical instruments may be precisely tracked throughout a surgical procedure.

### RELATED APPLICATIONS

This application claims the benefit of U.S. Provisional Patent Application No. 63/084,952, filed Sep. 29, 2020, the contents of which are incorporated herein in their entirety.

### GOVERNMENT INTEREST

This invention was made with United States government support under grant N00014-17-1-2566 from the Office of Naval Research. The U.S. government has certain rights in the invention.

### BACKGROUND

Pre-operative surgical planning involves the use of computer-aided imagery to superimpose multiple layers of the anatomy to determine defined surgical paths. Due to the dynamic status of anatomical regions during surgical procedures, real-time iterative surgical planning based on changing surgical tool locations is critical to improve the chances for a positive surgical outcome and to decrease surgical time. Further improvements in technology, for example, using smaller scale visually based electronics, have also provided the ability to create motion-tracking sensors that are integrated with surgical instruments to improve real time feedback for iteration of surgical operating and planning scenarios in confined cavity spaces. However, these surgical tool tracking approaches require a direct line of sight with bulky and expensive visual markers.

Currently, to assist surgeons in tracking surgical tools while performing minimally invasive surgical procedures, some commercially available systems use a combination of visual active and passive markers. These markers are visible impressions on surfaces strategically placed in an operating room, on the patient and on the surgical tools, which enable the optical tracking of the surgical tools with respect to the surgical site and rely on hand tracking for precision and accuracy. This approach not only augments the user's surgical technique, but also provides a path for pre-surgical planning. Other commercial systems utilizing computer vision (CV) have been designed to deploy additional haptic feedback to the intended user and help with fundamental surgical skills assessment and motion analysis tracking with assistance from augmented reality. Despite all these features, these systems still require a direct line of sight to the target area for surgical training effectiveness.

In some prior art examples, micro-electrical mechanical systems (MEMS) have been designed to generate simulated feedback in the form of haptic transduction as input for robotic assisted minimally invasive surgery. However, orientation error persists in these systems, and this increases user error, which negatively impacts surgical planning. In other instances of surgical tool tracking, an optical approach is used through utilizing imagery of the shape of a surgical instrument, along with a camera position that can be used to determine the position and orientation of an endoscopic instrument in an operating room. This approach localizes five degrees of freedom (i.e., two rotation angles around an access point, insertion depth, and rotation of the instrument around an axis). However, this method can have accuracy limitations as well as registration errors. This approach also can only be used for large-scale position tasks such as surgical navigation assistance tasks like proximity warnings. To address registration errors, other systems have used head mounted displays that relay select real time data to the user. These have been used in environments where the alignment of this imagery with the physical anatomy is feasible, but this approach provides a limited scope-of-view to the surgeon.

A promising alternative to CV-based approaches involves the use of inertial measurement units (IMUs). This is because an IMU can be programmed to transmit motion tracking data without the need for a direct line of sight. Technological advances with robotic surgery, smart instruments and flexible and stretchable electronics have brought about the use of IMUs in various applications. IMUs measure a body's force, angular rate, and orientation through a combination of accelerometers, gyroscopes, and magnetometers. While promising for surgical applications, further progress in the use of IMUs for continuous motion tracking depends on tighter integration with the equipment in the surgical environment.

### BRIEF DESCRIPTION OF THE DRAWINGS

FIG. 1 is a photographic image of an in situ instrumented scalpel of the present invention.

FIG. 2A is a schematic diagram showing the coordinate system of the 9-DoF IMU used in the present invention. FIG. 2B is a diagram showing the fusion of the data from all three sensing modalities for the three axes of the coordinate system into a single position.

FIGS. 3A-3D are schematic diagrams showing steps in the fabrication process of the flexible PCB-based IMU. FIG. 3E is a schematic diagram showing an alternate fabrication technique.

FIG. 4 shows a top schematic view of an example of a completed circuit traces on a flexible PCB.

FIG. 5 is an image showing the IMU in its native form from the manufacturer.

FIG. 6 is a diagram of the system architecture of the disclosed embodiments.

FIG. 7 is a series of images showing a proof-of-concept experiment of the disclosed embodiments.

FIG. 8 is a graph showing position readouts of the Z (pitch) axis during a proof-of-concept experiment.

FIG. 9 is a graph showing position readouts of the Z (pitch) axis compared to the X (roll) and Y (yaw) axes during the proof-of-concept experiment.

### SUMMARY OF THE INVENTION

Disclosed herein is an approach providing an instrumented hand-held tool for tracking a three-dimensional position of the tool. The instrumented tool leverages small scale electronics to enable real-time position capture for use in iterative procedure planning. By integrating a lightweight 9 degree-of-freedom (DoF) Inertial Measurement Unit with the hand-held tool, the method and system disclosed herein captures both spatial and temporal information of the movement of the tool without requiring a direct line-of-sight providing visual cues.

Data from the IMU is analyzed to determine the full range of motion during angular displacement for measurement and tracking. In preferred embodiments, the 9-DoF IMU is printed on a flexible film and attached to or integrated with the tool to allow precise tracking of the tool during user interaction.

Note that, although the invention is explained in the context of surgical tool, for example, a scalpel being used by a surgeon in a surgical field, the invention is applicable to any hand-held tool where it is desirable to provide precise tracking of the tool's position.

### DETAILED DESCRIPTION

The disclosed invention discloses an instrumented, hand-held tool and a method for tracking the tool. In certain embodiments, the tool may be, for example, a surgical tool using an inertial measurement unit printed on a flexible circuit, that is attached to or integrated with the surgical tool. The surgical tool may be, for example, a scalpel, wherein real time motion tracking and a measurement profile of a proposed surgical path is provided without the need for a direct line-of-sight between a tracking apparatus, for example, one or more cameras, and the tool. This enables and provides the capability for an un-occluded pre-surgical and iterative surgical path planning capability.

To date, a majority of IMU's rely on only an accelerometer and/or a gyroscope for precision tracking. By adding a magnetometer, the accuracy for the measurement of the tracking of the surgical tool is significantly improved. The invention integrates a 9 degree-of-freedom IMU with a surgical tool as an approach for tracking motion of the tool in a confined cavity space and relaying this data in real-time for an iterative approach for surgical planning.

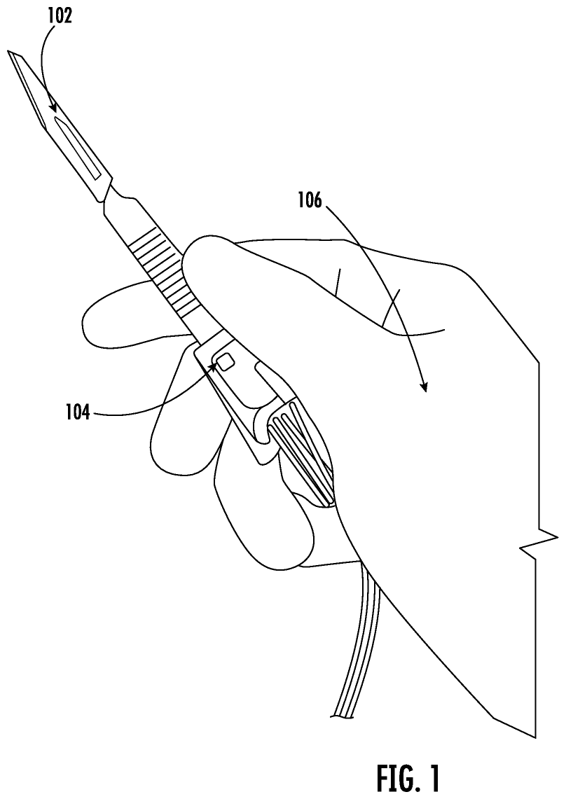

FIG. 1 is an image of a surgeon 106 holding a surgical tool 102 that has been fitted with an IMU 104 mounted on a flexible circuit board for real time tool tracking. The wires are the power and signal transmission lines. In preferred embodiments, a wireless version of the instrumented tool would be provided in which the tracking data is transmitted off-tool wirelessly and, further, wherein the tool is powered by an on-board battery.

In some embodiments, a surgical path may be defined for the surgeon 106 to follow with the instrumented tool 102. The instrumented tool can be localized, and the location presented on an orientation display monitor in full view of the surgeon 106. The motion and orientation of the instrumented tool may be presented with respect to a CAD model of the surgical field. The surgical path is then updated in accordance with this real time information to account for tissue dynamics. The additional sensing modality in the IMU not only improves measurement and location precision, but also addresses weight challenges associated with head mounted displays and overcomes the need for direct line-of-sight, without impeding any surgical technique guidelines. The approach allows the tracking of a full range of motion on the instrumented tool.

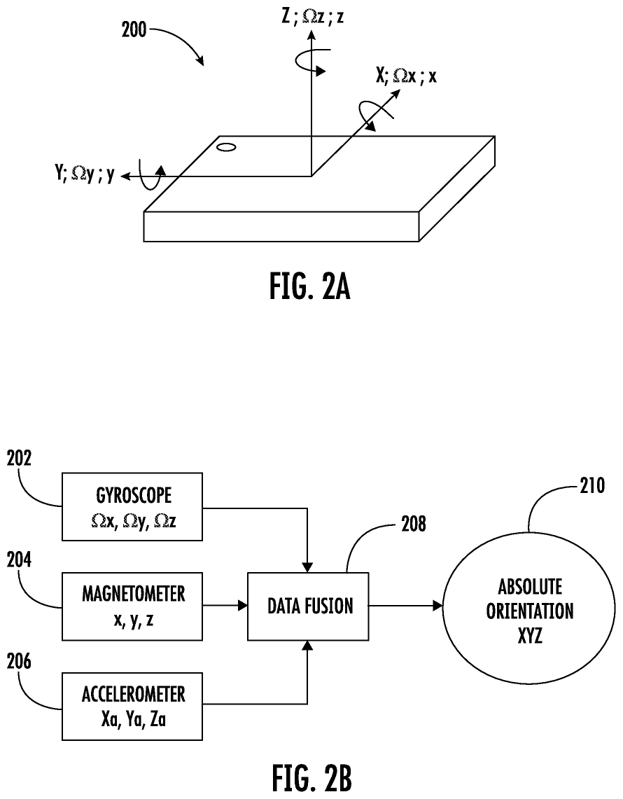

For tracking of the instrumented surgical tool, a 9-DOF IMU is used. The IMU, contains 3 internal triple-axis MEMS sensors, as shown in FIG. 2B, sensing position using three different sensing modalities. The first sensor is an accelerometer 206 that measures rotation and translation through an output of electrical capacitance when placed under mechanical stress. The accelerometer contains capacitive plates internally that are attached to a mechanical spring that moves internally as acceleration forces act upon the sensor. The movement of the plates relative to each other causes a capacitive change, that allows the acceleration to be determined. The second sensor is a gyroscope 202 that measures relative position to the earth's gravitational field by measuring the angular velocity from rotation around an axis and correlating that to a voltage to determine the position. The third sensor is a magnetometer 204 that measures proximity to the Earth's magnetic field by measuring a change in electrical current brought about by a change in magnetic flux density. The magnetic field affects the motion of the electrons, and this change can be used to determine the direction of the magnetic field.

An exemplary coordinate system 200 for the 9-DoF IMU is shown in FIG. 2A. Coordinate system 200 defines the output data axis for each sensor configuration, which, after data fusion, are further output as X (yaw), Y (roll) and Z (pitch) and is derived from Tait-Bryan angles, also known as nautical angles or Euler angles. As shown in FIG. 2B, the IMU 104 is provided with three sensing modalities: a gyroscope 202, a magnetometer 204 and an accelerometer 206, all of which provide position data with respect to three-axis coordinate system 200 for a total of 9-DoF. A data fusion algorithm 208 combines the position data from the three sensing modalities 202, 204, 206 and creates a single, three axis (X (Yaw), Y (roll), Z (Pitch)) absolute orientation 210 in the defined coordinate system 200.

In a clinical setting, the placement of surgical tool 102 in the center of the palm of surgeon 106, as shown in FIG. 1, can not only be done for ergonomic reasons but also to ensure that the surgeon's movements during surgery are not impeded. This is a position that is widely used when performing certain surgical techniques such as incision into cavity regions. The proposed surgical tool tracking method would have a customized flexible IMU 104 attached to the central pivot of the surgical tool 102 versus being positioned on either end of tool 102. This placement of flexible IMU 104 maintains the ergonomic attributes and does not impede a surgical technique with a direct line-of-sight requirement. Calibration of the IMU 104 to accurately track surgical tool 102 to provide a precise location after the calibration step is used to iterate the pre-planned surgical path once surgery begins.

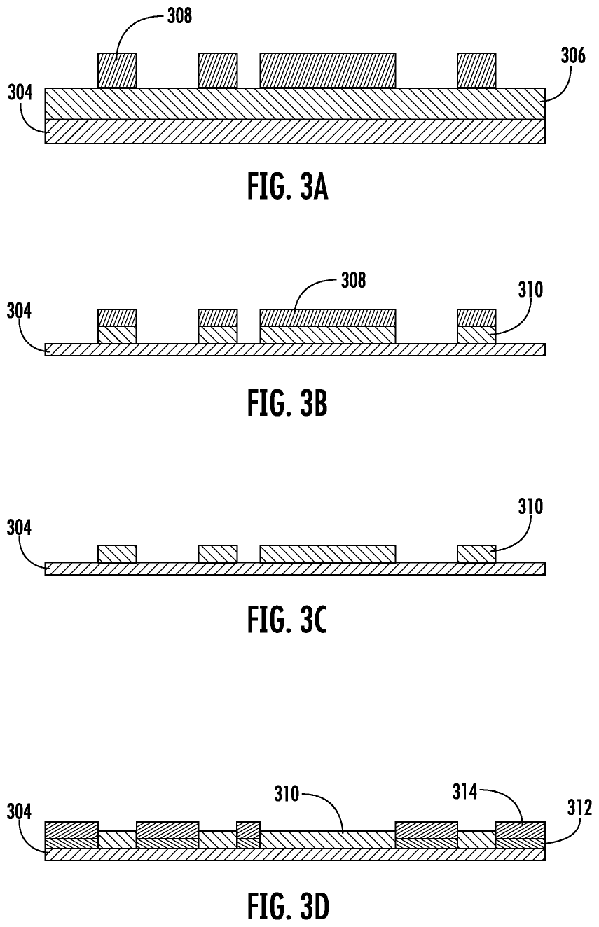

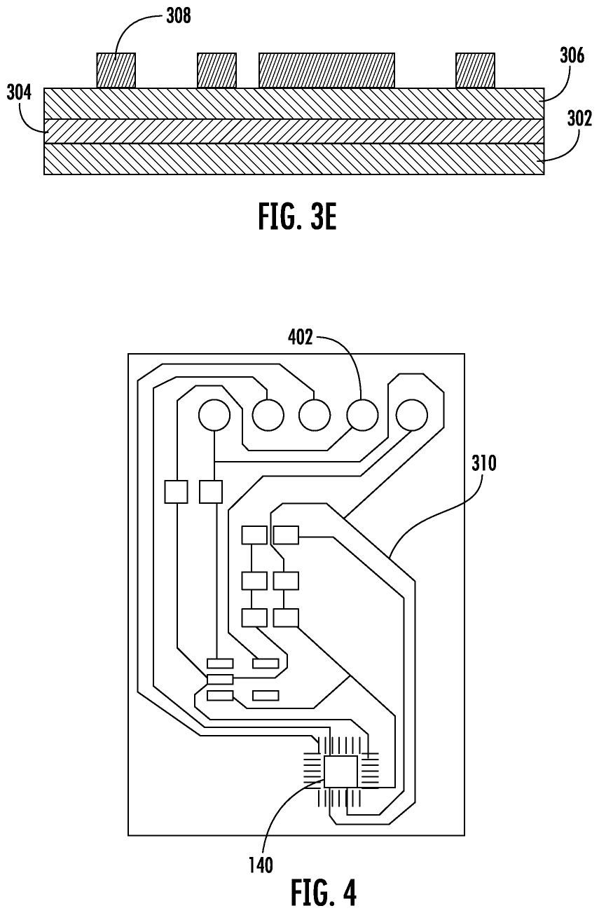

To ensure that the electronics are compatible with the contours of the surgical scalpel, the IMU and supporting circuitry are populated on a flexible printed circuit board (fPCB) as surface-mounted integrated circuits (IC). The fPCB is manufactured by combining flexible materials with IC electronics by a process shown in FIGS. 3A-3D to provide a thin and compliant construction with enhanced conformity.

The layout of the (fPCB), in one embodiment, is defined using off-the shelf design software and fabricated using a wax printer. FIG. 3A is a side view of the fPCB showing a laminated substrate comprising a layer of a polyimide film 304, for example, Kapton, having a layer of copper 306 deposited thereon. The copper may be deposited on the polyimide layer 304 by any know means. In one embodiment, the polyimide film 304 may be approximately 50 μm in thickness and the layer of copper 306 may be approximately 35 μm in thickness.

Masking ink 308 may then be deposited on the layer of copper to define the circuit pathways 310. The masking ink 308, in one embodiment, may be paraffin wax deposited by a wax printer. A solution of hydrogen peroxide, hydrochloric acid, and water are then mixed (2:1:1) to etch the sacrificial copper layer 306 exposed by the printed pattern 308. Removal of the wax ink by manual etching of the printed wax ink by a small scratch brush leaves a conductive copper circuit trace, as shown in FIG. 3C. Surface-mounted integrated circuit chips, including IMU 104, are then soldered to the board to complete the circuit. As shown in FIG. 3D, a protective layer of polyimide file 314 may be applied with a layer of adhesive 312 to protect the circuit. In one embodiment, the protective layer of polyimide file may be a layer of Kapton approximately 25 μm in thickness. The layer of polyimide film 314 may be provided with cutout areas to accommodate the surface mounted electronic components, but otherwise covers circuit pathways 310.

In a second embodiment shown in FIG. 3E, circuit pathways may be defined on both sides of the layer of polyimide film 304 by first depositing the layer of polyimide film 304 on a layer of copper 302. As with layer of copper 306, layer of copper 302 may be, in one embodiment, approximately 35 μm in thickness. The process for etching the circuit pathways in bottom layer of copper 302 is identical to that described above for etching the circuit pathways in copper layer 306. Circuit pathways defined in layers 302 and 306 may be connected by vias extending through the layer of polyimide film 304. This embodiment has the advantage of allowing fabrication of a smaller version of the flexible circuit.

An example of the completed circuit is shown in FIG. 4, showing IMU 140, circuit pathways 310 and wiring pads 402. Note that, in certain embodiments wherein the circuit may be provided with a means of wireless communication, for example, a Bluetooth chip, as well as an onboard means of power, wiring pads 402 may be eliminated. The exemplary circuit shown in FIG. 4 is then mated with the hand-held tool by mounting the circuit board on the hand-held tool, of providing an integration between the circuit board and the hand-held tool. Preferably, IMU 104 will be placed as close as possible to the central pivot point of all three axes of rotation of the hand-held tool.

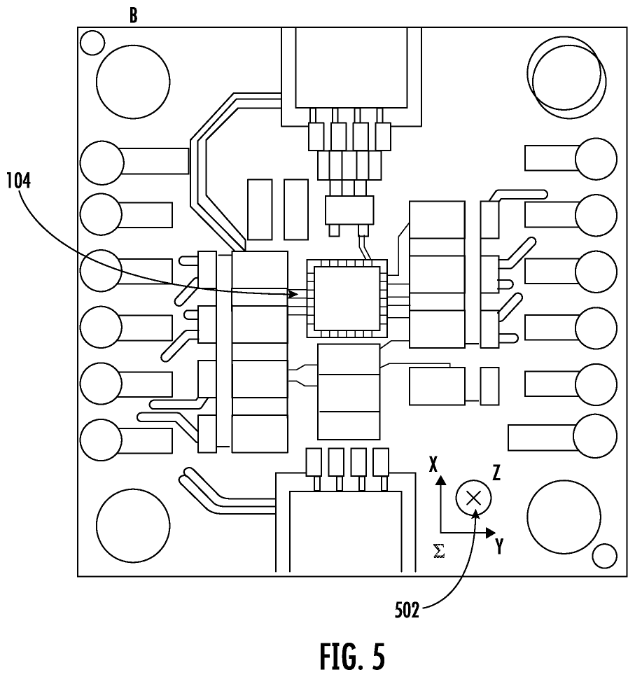

IMU 104 may be a commercial, off-the-shelf device provided on a hard circuit board. In one embodiment, IMU 104 may be part number ICM20948, manufactured by InvenSense, an example which is shown in FIG. 5, showing IMU 104 mounted on a hard printed circuit board which also shows the coordinate system 502. IMU 104 can later be removed from the hard circuit board and placed on the fPCB, as described above. Alternatively, a new IMU design for a hard printed or flexible PCB can be designed and manufactured for purposes of this invention.

To account for hard and soft iron distortions in the surgical field as well as any variations/noise, the magnetometer 204 has programmable digital filters that limit the range of measurement data in accordance with the manufacturer's specification. The gyroscope 202 and accelerometer 204 sensors, similarly per the manufacturer's specification, have a 1× average filter that smooths out the data during sampling.

The calibration is accomplished for each sensor on the hard circuit board per recommended manufacturing specifications. In embodiments using a commercially available IMU, the calibration procedure may be specified by the manufacturer of the IMU. For example, the following process may be used to calibrate the IvenSense IMU used in exemplary embodiments of this invention: To calibrate the accelerometer, one side of the board is moved along the 3 axes in both directions and is maintained in that position for 5 seconds. The gyroscope is calibrated, for example, in one embodiment by moving the board for 5 seconds and letting it rest on the table for 5 seconds. The magnetometer is calibrated by moving the board in a figure-8 style motion for a total of 5 times.

The internal runtime and background calibration for IMU 104 ensures that optimal performance of the sensor data is maintained with each output of absolute orientation data point (X, Y, Z) by having each data point be accompanied by a metric showing the calibration confidence for each reading. This metric is a measure of the calibration confidence from the data fusion for each data point as the measurement accuracy is made by the corresponding sensor.

In one embodiment, IMU 104 is programmed through a microcomputer, for example, an Arduino®, to provide position information through a custom API built to filter out the noise. In one embodiment, IMU 104 is set to send data at a 115,200 baud sampling rate based on the sensitivity range for each of the different sensors. The data fusion 208 from all three sensors provides data as a 3-D space absolute orientation 210, with respect to coordinate system 200 in which the X-axis represents yaw, the Y-axis represents roll, and the Z-axis represents pitch. In some embodiments, data fusion algorithm 208 may be provided by the manufacturer of the IMU 104, while, in other embodiments, the data fusion algorithm 208 may be developed separately and independently. Preferably, IMU 104 will provide the capability to output raw sensor data, in lieu of a single coordinate synthesized by the data fusion algorithm 208.

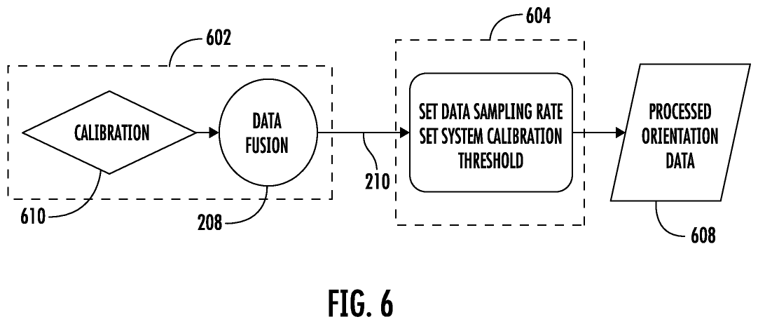

FIG. 6 shows the architecture of the software for processing the data generated by IMU 104. Portion 602, including the calibration function 610, described below, as well as the data fusion portion 208 is, in one embodiment, integrated with IMU 104. The output of portion 602 is the absolute position information 210 output by data fusion algorithm 208. Portion 604 is performed remotely from the instrumented tool and is implemented as custom software executing on the microcomputer, in preferred embodiments, an Arduino. Portion 604 handles the initial set up of IMU 104 and performs post-processing of the absolute position information 210 to output the processed orientation and movement data 608. MATLAB and C++ software on the Arduino are employed to analyze the data from IMU 104 and the results are output using an Arduino MKRZERO board.

For the proposed tracking method, IMU 104 provides a structured data set for absolute orientation that is obtained from the 9-DoF measurements. As one example, in the case of a tumor biopsy where an incision is made to perform the biopsy, this tracking method is meant to simulate motion during the actual moment that the surgeon, following a path of least resistance with a surgical tool (e.g., a scalpel) makes physical contact with the compact tissue. At this point of contact, there is limited further lateral motion with respect to the surgeon holding the scalpel in hand and the change in absolute orientation is related to a pivot at the wrist to make a vertical incision into a tissue with the scalpel. Due to this technique, this scenario can be modeled and tracked with the system disclosed herein. Because of the small cavity of the body within which the surgeon must operate, the accuracy of knowing this precise location, which is then communicated to other systems used in surgical path navigation, is critical.

For data analysis, the overall volume RMS distance error is used to analyze the error after multiple runs and is used to correct for the error. Data points from each run are compared with each other over time to determine the difference, ε_{RMS}, between the measured positions, rₘ, and their corresponding reference position, rᵣ, as εᵢ=rᵣᵢ−rₘ_{ᵢ} for each data point, i. For this analysis, the first run is taken as the baseline run and the positions are obtained from their angular displacement, in degrees, based on the full range of motion. Then:

$${ɛ}_{RMS} = \sqrt{\frac{1}{N} {\sum }_{i = 1}^{N} ( {ɛ}_{i} \cdot {ɛ}_{i} )} \qquad ( 1 )$$

A proof-of-concept experiment was conducted and will now be described. The IMU 104 is attached to a surgical scalpel 102, as shown in FIG. 1, via the fPCB. The purpose of mounting the circuit to the scalpel is to simulate the clinical conditions required for actual surgical applications. Tracking of the tool 102 will allow real time iterative surgical planning to occur.

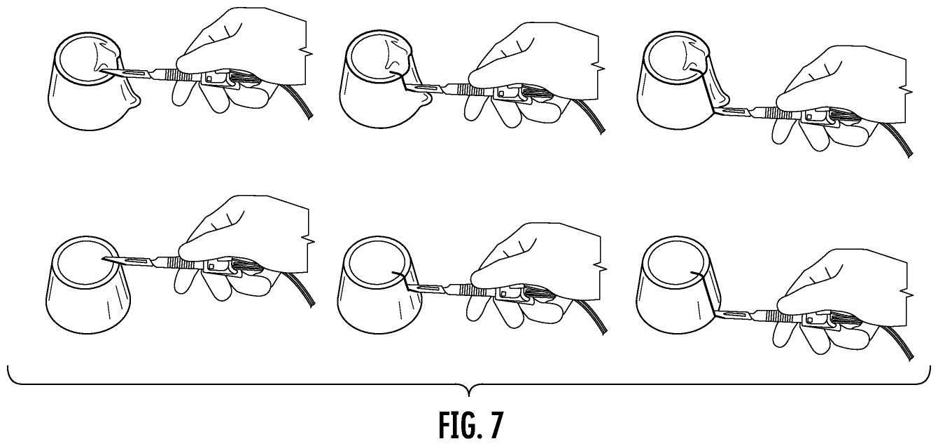

The motion of the instrumented tool is tracked during its initial contact with, and via an incision cut on, a gelatin based biomimetic substrate, as shown in FIG. 7, based on a pre-planned surgical vertical path. The gelatin is a type of hydrogel that can be used as 3D tissue scaffolds due to tissue-like mechanical properties such as elasticity, stiffness and geometry and thus have been used as engineered tissue to simulate muscle-like structures. Hydrogels have further been used to 3D bio print systems, such as aortic valves, while maintaining mechanical properties such as ultimate strength and peak strain and maintaining the tensile biomechanics comparable to actual muscle tissue. The IMU is attached to the center of the scalpel as an approximate pivot point for tracking absolute orientation data. Three incisions are made in positive and negative (up and down vertical) motions in the Z-axis (pitch) into the gelatin muscle tissue substrate. These three incisions are made in new locations each time. The movement of the sensor translates to the angular displacement in terms of the Z-axis for the measurement.

In addition, the flexible IMU 104 is then wrapped around the scalpel with the overall scalpel position similar to the printed circuit board to simulate tissue response during scalpel motion for three incisions into the gelatin using a proposed application setup of the flexible IMU 104.

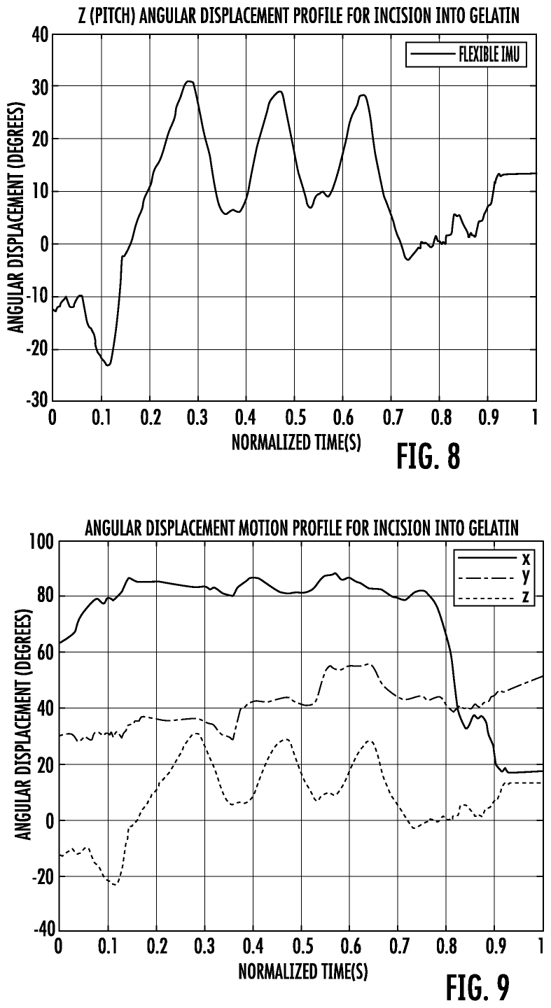

The motion of the flexible IMU attached to the surgical scalpel shown in graph form in FIG. 8 and shows three different peaks signifying the angular displacement of the scalpel each time the scalpel enters and leaves the gelatin muscle-like substrate. This motion is expected to be reflected only in the pitch (Z) axis. The motion profile for all three axes during the three incisions is shown in FIG. 9. In this motion tracking of all three orientation axes, the Z-axis (pitch) shows a change in angular displacement consistent with the three incisions with each incision peak signifying that respective travel. The angular displacement in the X-axis (roll) axis and the Y-axis (yaw) is expected to be relatively linear during the three incision peaks into the gelatin muscle substrate, as the only movement made by an experienced surgeon would happen in the Z-axis. However, due to the lack of surgical experience of the experimenter, limited angular displacement occurs during the incision into the gelatin muscle substrate, as indicated by the non-flat profile of the tracked angular displacement in the X (Yaw) and Y (Roll) axes during the three peaks associated with the incisions found in the Z-axis in FIG. 8.

It is also important to note that for the proposed experimental setup, there is no tracking of the lateral linear motion of the scalpel and only the absolute orientation of an object at its pivot is tracked. This setup represents the actual surgical incision cuts made manually in the surgical field. The absolute orientation picked for surgical tool tracking is reflective of the type of iterative surgical planning that is hard to predict and would require further analysis as a base comparison between the proposed approach of a flexible IMU attached to a surgical tool and an industrial tracking method that utilizes a commercial product with optical tracking capabilities as a benchmark. Furthermore, the decision to take only calibration data points having an acceptable calibration metric associated therewith enabled a consistent output for the three axes with the data not being affected by magnetic distortion.

The ability of a 9-DoF IMU to track measurement motion related to a surgical tool without requiring a direct line of sight has been demonstrated for absolute orientation tracking. Real time surgical tool locations are necessary to help close the feedback loop of a pre-surgical planned path to generate new surgical paths as the surgical procedure is carried out. Current approaches that rely on optical trackers to track the user and the surgical tools in this environment through a direct line-of-sight can hinder a full understanding of surgical techniques as the user would have to accommodate the constraint. A 9-DoF IMU is integrated onto the test platform to demonstrate its utility in an angular motion measurement representative of absolute orientation by engaging in one of its axes at a time to track the motion. The IMU and data analysis allows obtaining absolute orientation data for a tracked surgical instrument. This approach is then used to move and track a scalpel, attached with a customized flexible IMU, through a gelatin based biomimetic substrate when a series of incisions is made in the absolute orientation frame. In other embodiments, the capability of tracking as a flexible IMU film could be useful to integrate with other minimally invasive surgical approaches like gloves and robotic systems to provide additional autonomy with surgical planning.

This approach can be applied to other applications, such as tracking a surgical tool along a defined and known path to determine real time error and its effects on proper surgical planning. Furthermore, the ability to link to motion when interacting with other tissue substrates with different mechanical properties allows for integration with system analysis and augmented reality approaches to surgery. Still further, the flexible IMU can be embedded in other tools and instruments for non-surgical work that require precision location tracking.

1. A device comprising:
an inertial measurement unit (IMU); and
a flexible circuit board defining a support circuit for the IMU, the IMU being mounted on the flexible circuit board.

2. The device of claim 1 wherein the flexible circuit board comprises copper circuit pathways defined on a temperature-stable polyimide film and a plurality of surface-mounted integrated circuits.

3. The device of claim 2 wherein the polyimide film is Kapton.

4. The device of claim 2 further comprising a protective layer of a polyimide film on the defined circuit, the polyimide film having cutouts for the plurality of surface-mounted integrated circuits.

5. The device of claim 1 wherein the IMU senses positional data using 9 degrees of freedom.

6. The device of claim 1 further comprising means for communicating data generated by the IMU off-board.

7. The device of claim 6 further comprising:
a processor;
software, executing on the processor for performing the functions of:
receiving data indicative of a movement of the IMU in three-dimensional space;
receiving a metric indicating the validity of the data;
filtering the received data; based on the received metric; and
outputting filtered data indicative of movements of the IMU in three dimensional space.

8. The device of claim 6 wherein the software performs the further function of performing calibration and set-up functions for the IMU.

9. The device of claim 7 further comprising:
a hand-held tool having the flexible circuit board mounted thereon or integrated therewith.

10. The device of claim 9 wherein the filtered data output by the software is indicative of movements of the hand-held tool along axes representing pitch, yaw and roll.

11. The device of claim wherein the support circuitry defined on the flexible circuit board include components supporting wireless communication of data generated by the IMU off-board.

12. The device of claim 10 wherein the hand-held tool is a surgical instrument.

13. A method of fabricating an instrumented hand-held tool comprising:
providing a layer of a polyimide film;
depositing a layer of copper on the layer of temperature-stable polyimide film;
depositing an etching mask defining a plurality of circuit traces on the layer of copper;
etching the exposed areas of the copper layer;
removing the etching mask; and
mounting one or more surface-mounted integrated circuits on the circuit traces, the one or more integrated circuits including an inertial measurement unit (IMU).

14. The method of claim 13 wherein the layer of polyimide film comprises a layer of temperature-stable polyimide film.

15. The method of claim 14 wherein the layer of temperature-stable polyimide film comprises a layer of Kapton approximately 50 microns in thickness.

16. The method of claim 13 wherein the layer of copper is approximately 35 microns in thickness.

17. The method of claim 13 wherein the etching mask comprises paraffin wax deposited by a wax printer.

18. The method of claim 13 further comprising:
mounting the flexible circuit board on a hand-held tool such that the IMU is located at an approximate center of rotation of roll, pitch and yaw axes of the hand-held tool.

19. The method of claim 17 wherein the IMU exports data indicative of a fusion of positions of multiple sensing modalities to an off-board processor.

20. The method of claim 18 further comprising:
providing a processor executing software, the processor receiving data from the IMU, the software performing the functions of
filtering the data; and
outputting data indicative of movements of the hand-held tool along axes representing pitch, yaw and roll of the hand-held tool.
